(19) United States
(12) Patent Application Publication
(10) Pub. No.: US 2005/0118109 A1
(43) Pub. Date: Jun. 2, 2005

(54) Method for treating lung diseases associated with ventilation-perfusion mismatches

(76) Inventors: Lutz-Henning Block, Munich (DE); Rolf Ziesche, Sommerein (AT); Ventzislav Petkov, Wein (AT)

Correspondence Address: WEBB ZIESENHEIM LOGSDON ORKIN & HANSON, P.C., 700 KOPPERS BUILDING, 436 SEVENTH AVENUE, PITTSBURGH, PA, 15219, US

(21) Appl. No.: 10/898,143

(22) Filed: Jul. 23, 2004

Related U.S. Application Data
(60) Provisional application No. 60/489,744, filed on Jul. 24, 2003.

(51) Int. Cl. A61K 38/10; A61K 38/08; A61K 31/4745; A61K 31/573; C07K 7/08; C07K 7/06

(52) U.S. Cl. 424/45; 514/12; 514/13; 514/14; 514/15; 530/324; 530/325; 530/326; 530/327; 514/291; 514/171; 514/634

(57) ABSTRACT

The present invention relates to pharmaceutical compositions and methods for the prevention and/or treatment of lung diseases or disorders including the bronchial tree, in an animal or human, such as chronic obstructive pulmonary disease (COPD), and diseases related to or optionally associated with COPD-like lung disorders caused by ventilation-perfusion mismatches preferably in context with chronic bronchitis. The treatment includes administration of pharmaceutical compositions comprising vasoactive intestinal peptide (VIP), pituitary adenylate cyclase-activating polypeptide (PACAP), and biologically active analogues thereof, which comprise highly conservative sequence tracks.

### CROSS REFERENCE TO RELATED APPLICATIONS

The present invention claims priority to U.S. Provisional Application No. 60/489,744, filed Jul. 24, 2003, which is incorporated by reference herein.

### BACKGROUND OF THE INVENTION

1. Field of the Invention

The present invention relates to pharmaceutical compositions and methods for the prevention and/or treatment of lung diseases and disorders, including the bronchial tree, caused by or associated with ventilation-perfusion mismatches (V/Q-mismatches), preferably in conjunction with chronic bronchitis, such as chronic obstructive pulmonary disease (COPD), and diseases associated with COPD.

2. Description of the Related Art

Chronic obstructive pulmonary disease (COPD) is a term that encompasses a group of chronic lung conditions characterized by obstruction of the airways of the lungs. COPD generally includes two major breathing diseases: chronic (obstructive) bronchitis and emphysema. Both breathing diseases make breathing difficult and cause breathlessness. COPD may be, but not necessarily, accompanied by primary pulmonary hypertension (PPH) or secondary pulmonary hypertension (SPH).

Chronic bronchitis is an inflammatory progressive disease that begins in the smaller airways within the lungs and gradually advances to larger airways. It increases mucus production in the airways and increases the occurrence of bacterial infections in the bronchial tree, which, in turn, impedes airflow. This chronic inflammation induces thickening of the walls of the bronchial tree leading to increased congestion in the lungs, which results in dyspnea. By definition, chronic bronchitis refers to a productive cough for at least three months of each of two successive years for which other causes have been ruled out.

Emphysema underlies COPD and damages and destroys lung architecture with enlargement of the airspaces and loss of alveolar surface area. Lung damage is caused by weakening and breaking of the air sacs, i.e., alveoli, within the lungs. Several adjacent alveoli may rupture, forming one large space instead of many small ones. Larger spaces can combine into an even bigger cavity, called a bulla. As a result, natural elasticity of the lung tissue is lost, leading to overstretching and rupture. There also is less pull on the small bronchial tubes, which can cause them to collapse and thus obstruct airflow. Air that is not exhaled before new air is inhaled gets trapped in the lungs, leading to shortness of breath. The sheer effort it takes to force air out of the lungs when exhaling can be exhausting.

COPD is always accompanied by bronchial obstruction. Thus, the most common symptoms of COPD include shortness of breath, chronic coughing, chest tightness, greater effort to breathe, increased mucus production and frequent clearing of the throat. Patients are unable to perform their usual daily activities. Independent development of chronic bronchitis and emphysema is possible, but most people with COPD have a combination of the two disorders. Both conditions decrease the ability of the lungs to take in oxygen and remove carbon dioxide. Although the airway limitation associated with COPD has often been regarded as irreversible, it has been shown that the airway limitation is partially reversible.

COPD prevalence increases with age, but there is a dramatic synergy with smoking such that smokers have higher COPD prevalence and mortality and lung function losses. A smoker, therefore, is ten times more likely than a non-smoker to die of COPD. When inhaled, cigarette smoke paralyzes the microscopic hairs, i.e., cilia, which line the bronchial tree. Irritants and infectious agents caught in the mucus remain in the bronchial tree rather than being swept out by the cilia. This can inflame bronchial membranes, eventually resulting in chronic obstruction. Other indoor and outdoor air pollutants may damage the lungs and contribute to COPD. Thus, long-term cigarette smoking is the predominant risk factor for COPD, accounting for 80 to 90% of the risk for developing the disease, yet only about 15% of all smokers actually develop COPD severe enough to cause symptoms. Other risk factors for COPD are heredity, second-hand smoke, air pollution, and a history of frequent childhood respiratory infections.

COPD is often misdiagnosed as asthma or remains undiagnosed in its mild and moderate stages. It has been estimated that up to 75% of people suffering from COPD are undiagnosed. The medical histories of COPD and asthma are distinctly different with different etiologies and treatments. Some of the differences between COPD and asthma are: (1) asthma patients typically have an age of onset earlier in life, whereas COPD patients tend to be older; (2) there is no direct link between asthma and smoking, whereas COPD is strongly associated with smoking; (3) dyspnea, or shortness of breath on exertion, is far more common in COPD than in asthma; (4) COPD symptoms are progressive, whereas asthma symptoms are more episodic and stable over time; and (5) inflammation is central to asthma, whereas the inflammatory role in COPD is far less clear.

Another lung disease that often results in COPD is acute (adult) respiratory distress syndrome (ARDS). ARDS is a severe injury to most or all of both lungs and is a life-threatening condition. ARDS is characterized by a rapid onset of progressive malfunction of the lungs, especially with regard to the ability to take in oxygen, and typically is associated with the malfunction of other organs of the body. The condition is associated with extensive lung inflammation and accumulation of fluid in the alveoli which leads to low oxygen levels in the lungs. ARDS is associated with diffuse pulmonary microvascular injury resulting in increased permeability and non-cardiogenic pulmonary edema. To date, there are no specific pharmacological interventions of proven value for the treatment of ARDS. Although corticosteroids and prostaglandin E1 have been widely used clinically, studies have failed to show any benefit in outcome, lung compliance, pulmonary shunts, chest radiograph, severity score or survival. A number of new treatment approaches for ARDS is being explored, such as the administration of inhibitors of tumor-necrosis-factor alpha (TNF-α) and phosphodiesterase. Presently no measures are known to prevent ARDS.

Alveoli of a healthy lung typically look like a bunch of grapes. Ventilation is defined as the movement of air inside and outside of these alveoli. Each alveolus is surrounded by small blood vessels, i.e., capillaries. Perfusion is defined as the movement of blood through these vessels. The area where the alveoli and capillary blood vessels meet is where the exchange of oxygen and carbon dioxide occurs. When the lungs are affected by inflammation, for example because of chronic bronchitis, there is a decrease in airflow and permanent destruction of the alveoli in the lungs. Over time this creates areas where there remains a blood supply but without sufficient alveoli. This produces a ventilation-perfusion mismatch (V/Q-mismatch). V (ventilation) is defined as the rate of oxygen delivery to the alveoli and carbon dioxide elimination from the alveoli into expired air, and Q (perfusion) is defined as the rate of oxygen transport from the alveoli into the blood and carbon dioxide elimination from the blood into the alveoli. As a consequence of this V/Q-mismatch, there is less surface area for oxygen to get from the lungs and into the blood and for carbon dioxide to get from the blood and into the lungs to be exhaled. This can reach a point where the amount of oxygen in the blood is low (hypoxemia) and concomitantly the amount of carbon dioxide in the blood is relatively high (hypercarbia).

When the ratio of ventilation to perfusion, referred to as the V/Q ratio, is 1, the amount of blood circulating through the peripheral pulmonary arteries and alveolar capillaries matches the ventilated bronchioles and alveoli. A V/Q ratio of 1, therefore, is an indication of optimum pulmonary diffusion of oxygen and carbon dioxide. Thus, in contrast to ventilation parameters that are used for assessment of obstructive bronchial ventilation in chronic obstructive bronchitis and emphysema, the V/Q ratio is able to provide an immediate assessment of pulmonary circulation and diffusion capacity of the lungs.

Chronic inflammation of the peripheral lungs that includes both peripheral bronchi and alveoli may be accompanied by a decrease of the optimal ratio between ventilation and perfusion, even without an obstructive ventilation pattern typically required for a diagnosis of COPD. Thus, chronic inflammation of the peripheral lungs may worsen pulmonary gas exchange directly by decreasing the peripheral pulmonary blood flow in inflamed peripheral pulmonary tissues, causing a V/Q-mismatch that is independent of any bronchial obstruction. This results in a decreased diffusion capacity of the lung as reflected by a lower oxygen uptake (paO2), and an increase of the arterial-alveolar oxygen difference (AaDO2). The optimal overall V/Q ratio for a healthy lung system is between about 0.8 and 1.0. V/Q ratios lower than 0.8 and higher than 1.0 typically are regarded as in the pathological range.

Chronic bronchitis, with or without V/Q-mismatch, is rarely regarded as an indication for therapeutic intervention. This is due largely to the side effects of chronic anti-inflammatory treatments, such as oral or inhalative glucocorticoid application. It is clear, however, that any inflammatory condition of the peripheral lungs may become functionally relevant and thus may require eventual treatment, even without any demonstration of bronchial obstruction. Any treatment able to diminish a V/Q-mismatch would be beneficial for all types of chronic bronchitis, even those types that do not meet the criteria for a diagnosis of COPD. Thus, COPD may be, but not necessarily, associated with a V/Q-mismatch, whereas a V/Q-mismatch may be observed in ventilation disorders of the bronchial system that are not associated with an obstructive component.

Clinical development of COPD is typically described in three stages, as defined by the American Thoracic Society:

Stage 1: Lung function, as measured by forced expiratory volume in one second, or FEV1, is greater than or equal to 50 percent of predicted normal lung function. There is minimal impact on health-related quality of life. Symptoms may progress during this stage, and patients may begin to experience severe breathlessness, requiring evaluation by a pulmonologist.

Stage 2: FEV1 lung function is 35 to 49 percent of predicted normal lung function, and there is a significant impact on health-related quality of life.

Stage 3: FEV1 lung function is less than 35 percent of predicted normal lung function, and there is a profound impact on health-related quality of life.

According to the Annual World Health Report of the World Health Organization (WHO), about 600 million people suffer from COPD worldwide, with about three million people dying from the disease each year. Although there is no cure for COPD, medications and treatment typically prescribed for people with COPD include: fast-acting beta2-agonists, such as albuterol; anticholinergic bronchodilators, such as ipratropium bromide; theophylline derivatives; long-acting bronchodilators; inhaled or oral corticosteroids; antibiotics that are given at the first sign of a respiratory infection to prevent further damage and infection in diseased lungs; expectorants that help loosen and expel mucus secretions from the airways, and may help make breathing easier; lung transplantation, which may be an option for people who suffer from severe emphysema; lung volume reduction surgery; or treating alpha-1-antitrypsin (AAT) deficiency emphysema, which requires life-long AAT replacement therapy, such as gene therapy to substitute for the AAT deficiency.

Thus, although there are various treatment options and medications for treating lung diseases and disorders, there exists a need to provide a method of preventing and/or treating lung diseases or disorders which is efficacious and does not have some of the debilitating side-effects of current treatment options.

### SUMMARY OF THE INVENTION

The present invention provides a method of preventing and/or treating a lung disease or disorder in an animal or human in need thereof which is associated with a pathologically effective ventilation-perfusion mismatch (V/Q-mismatch) that may be, but not necessarily, associated with chronic obstructive pulmonary disorder (COPD), by administering to the animal or human in unit dosage form a therapeutically effective amount of a pharmaceutical composition that contains a polypeptide of about 10 to 38 naturally occurring amino acid residues, and preferably a polypeptide of about 18 to 38 naturally occurring amino acid residues having an N-terminal starting sequence consisting of His-Ser-Asp-X¹-X²-Phe-Thr-Asp-, wherein X¹ and X² may be any naturally occurring amino acid residue, and wherein the polypeptide contains the conservative sequence track Arg-Lys-Gln-Met-Ala-Val-Lys-Lys-Tyr-Leu.

The polypeptide of about 10 to 38 naturally occurring amino acid residues can include, without limitation, the following amino acids: Arg-Lys-Gln-Met-Ala-Val-Lys-Lys-Tyr-Leu; Phe-Thr-Asp-X¹-X²-X³-X⁴-X⁵-Arg-Lys-Gln-Met-Ala-Val-Lys-Lys-Tyr-Leu-Asn-Ser-Ile-Leu-Asn; Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn; Phe-Thr-Asp-Ser-Tyr-Ser-Arg-Tyr-Arg-Lys-Gln-Met-Ala-Val-Lys-Lys-Tyr-Leu; His-Ser-Asp-X¹-X²-Phe-Thr-Asp-X³-X⁴-X⁵-X⁶-X⁷-Arg-Lys-Gln-Met-Ala-Val-Lys-Lys-Tyr-Leu; His-Ser-Asp-Ala-Val-Phe-Thr-Asp-Asn-Tyr-Thr-Arg-Leu-Arg-Lys-Gln-Met-Ala-Val-Lys-Lys-Tyr-Leu; His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu; His-Ser-Asp-X¹-X²-Phe-Thr-Asp-X³-X⁴-X⁵-X⁶-X⁷-Arg-Lys-Gln-Met-Ala-Val-Lys-Lys-Tyr-Leu-X⁸-X⁹-X¹⁰-X¹¹ (-X¹²); His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn (VIP); His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu (PACAP-27); His-Ser-Asp-X¹-X²-Phe-Thr-Asp-X³-X⁴-X⁵-X⁶-X⁷-Arg-Lys-Gln-Met-Ala-Val-Lys-Lys-Tyr-Leu-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²; or His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-Gly-Lys-Arg-Tyr-Lys-Gln-Arg-Val-Lys-Asn-Lys (PACAP-38), wherein X¹-X²² is any naturally occurring amino acid residue.

Lung diseases or disorders that can be prevented and/or treated according to the method of the present invention can include, without limitation, COPD, preferably in conjunction with chronic bronchitis; chronic bronchitis not associated with COPD; lung disease not associated with pulmonary or arteriolar hypertension; or ARDS. Forms of COPD can include, without limitation, chronic bronchitis showing significant ventilation obstruction, pulmonary emphysema or chronic cough, such as smoker's cough.

The pharmaceutical composition of the present invention can be administered daily, wherein such treatment results in an improvement of the FEV1 value of more than 15% and an improvement of the paO2 value of more than 35% after about 3 months of daily treatment. The pharmaceutical composition can contain the effective polypeptide in a stabilized form, such as a pegylated form or in the form of a fusion protein, wherein the concentration of the effective polypeptide is between about 10 to 2000 μg/l, preferably between about 50 to 1500 μg/l, and most preferably between about 100 to 1000 μg/l. The pharmaceutical composition can be an aerosol, preferably based on a sodium chloride solution, which can be inhaled by a patient. The pharmaceutical composition thus can be used as a medicament or as a diagnostic means to evaluate pathological conditions in an individual.

The present invention also provides a method for improving or recovering the general state of health in an animal or human which has been reduced by chronic bronchitis associated with a pathologically effective ventilation-perfusion (V/Q)-mismatch without significant obstructive ventilation disorder, by administering to the animal or human a pharmaceutically effective amount of a composition containing vasoactive intestinal peptide (VIP), pituitary adenylate cyclase-activating polypeptide (PACAP), or an analogous polypeptide having the same biological activity of VIP or PACAP.

The pharmaceutical compositions of the present invention can contain one or more pharmaceutically acceptable carriers. Suitable carriers, such as liquid carriers, are well known in the art and can include, without limitation, sterile water, saline, aqueous dextrose, sugar solutions, ethanol, glycols and oils, such as petroleum, animal, vegetable, or synthetic oils. Exemplary oils can include, without limitation, peanut oil, soybean oil or mineral oil.

An effective therapeutic amount of the pharmaceutical composition of the present invention can be between about 5 ng to 28 μg/kg body weight, preferably between 15 ng to 25 μg/kg body weight, and most preferably between about 1 to 25 μg/kg body weight.

The present invention further provides a pharmaceutical composition that can be inhaled by the patient in the form of an aqueous solution containing a water-soluble peptide having the biological and pharmacological activity of the above-described VIP, PACAP and related analogues, variants, derivatives, homologues and the like. The pharmaceutical composition of the present invention preferably is in the form of an aerosol for inhalation, especially when the patient is suffering from chronic bronchitis. Administration by nasal spray techniques also are suitable.

The concentration of the particular peptide contained in the aqueous solutions can vary between about 10 to 2000 μg/L solution, preferably between about 50 to 1500 μg/L, and most preferably between about 100 to 1000 μg/L. If the particular peptide compound is in a stabilized form, the concentration, as well as the overall dosage of the peptide compound, can be decreased. The peptides or polypeptides can be administered via inhalation about 3 to 4 times a day for about 3 to 20 minutes, and preferably about 5 to 10 minutes, according to the severity of the disease and the potency of the compounds administered.

In a further embodiment of the present invention, the pharmaceutical compounds can be administered to an animal or human in need thereof in combination with other pharmaceutically effective compounds, e.g., fast-acting beta2-agonists, such as albuterol; anticholinergic bronchodilators, such as ipratropium bromide; long-acting bronchodilators; inhaled or oral corticosteroids, antibiotics, or antiproliferative compounds, such as D-24851, Imatinib mesylate or guanylhydrazone CNI-1493.

### BRIEF DESCRIPTION OF THE DRAWINGS

FIG. 1 shows an overview of four pilot patients, three suffering from COPD, and one suffering from chronic bronchitis with V/Q-mismatch. The latter one did not show any sign of bronchial obstruction, such as in COPD;

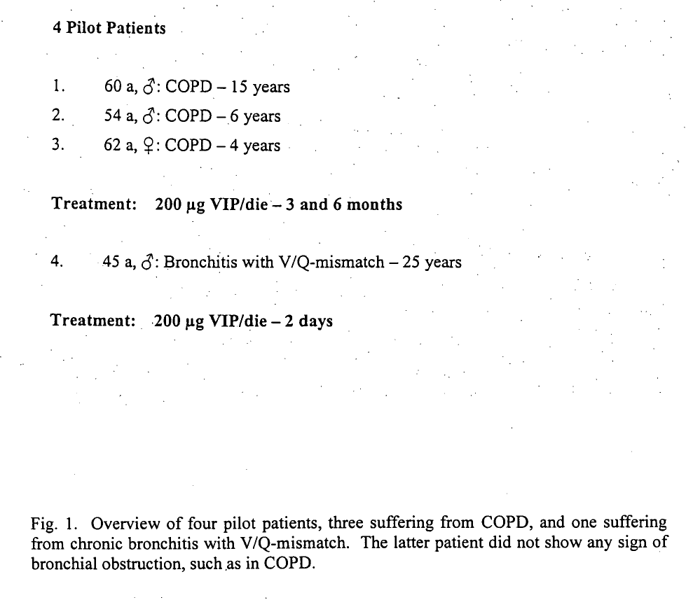

FIG. 2 shows the lung volumes of patient No. 1, namely the (expiratory) vital capacity (VC), the forced expiratory volume in one second (FEV₁), the total lung capacity (TLC), the residual volume (RV), and the peak expiratory flow (PEF);

FIG. 3 shows the blood gas analysis (paO2: partial arterial oxygen pressure; paCO2: partial arterial carbon dioxide pressure; and AaDO2: arterial-alveolar oxygen pressure difference) of patient No. 1 at baseline and three months later;

FIG. 4 shows a six minute walking distance of patient No. 1 at baseline and three months later;

FIG. 5 shows lung function parameters before and after six months of inhalation of VIP;

FIG. 6 shows FEV1 (forced expiratory volume in one second) and PEF (peak expiratory flow); blood gas analysis (paO2: partial arterial oxygen pressure; paCO2: partial arterial carbon dioxide pressure; AaDO2: arterial-alveolar oxygen pressure difference) of patient No. 2 at baseline and six months later;

FIG. 7 shows the lung volume of patient No. 3, namely the (expiratory) vital capacity (VC), the forced expiratory volume in one second (FEV₁), the total lung capacity (TLC), the residual volume (RV), and the peak expiratory flow (PEF);

FIG. 8 shows the blood gas analysis (paO2: partial arterial oxygen pressure; paCO2: partial arterial carbon dioxide pressure; AaDO2: arterial-alveolar oxygen pressure difference) of patient No. 3 at baseline and six months later; and

FIG. 9 shows the original lung function analysis of patient No. 4 prior to the inhalation of VIP.

### DETAILED DESCRIPTION OF THE PREFERRED EMBODIMENTS

The present invention provides a method of preventing and/or treating a lung disease or disorder in an animal or human in need thereof which is associated with a pathologically effective ventilation-perfusion mismatch (V/Q-mismatch) that may be, but not necessarily, associated with chronic obstructive pulmonary disorder (COPD), by administering to the animal or human in unit dosage form a therapeutically effective amount of a pharmaceutical composition that contains a polypeptide of about 10 to 38 naturally occurring amino acid residues, and preferably a polypeptide of about 18 to 38 naturally occurring amino acid residues having an N-terminal starting sequence consisting of His-Ser-Asp-X¹-X²-Phe-Thr-Asp-, wherein X¹ and X² may be any naturally occurring amino acid residue, and wherein the polypeptide contains the conservative sequence track Arg-Lys-Gln-Met-Ala-Val-Lys-Lys-Tyr-Leu.

The polypeptide of about 10 to 38 naturally occurring amino acid residues can include, without limitation, the following amino acid sequences: Arg-Lys-Gln-Met-Ala-Val-Lys-Lys-Tyr-Leu; Phe-Thr-Asp-X¹-X²-X³-X⁴-X⁵-Arg-Lys-Gln-Met-Ala-Val-Lys-Lys-Tyr-Leu-Asn-Ser-Ile-Leu-Asn; Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn; Phe-Thr-Asp-Ser-Tyr-Ser-Arg-Tyr-Arg-Lys-Gln-Met-Ala-Val-Lys-Lys-Tyr-Leu; His-Ser-Asp-X¹-X²-Phe-Thr-Asp-X³-X⁴-X⁵-X⁶-X⁷-Arg-Lys-Gln-Met-Ala-Val-Lys-Lys-Tyr-Leu; His-Ser-Asp-Ala-Val-Phe-Thr-Asp-Asn-Tyr-Thr-Arg-Leu-Arg-Lys-Gln-Met-Ala-Val-Lys-Lys-Tyr-Leu; His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu; His-Ser-Asp-X¹-X²-Phe-Thr-Asp-X³-X⁴-X⁵-X⁶-X⁷-Arg-Lys-Gln-Met-Ala-Val-Lys-Lys-Tyr-Leu-X⁸-X⁹-X¹⁰-X¹¹ (-X¹²); or His-Ser-Asp-X¹-X²-Phe-Thr-Asp-X³-X⁴-X⁵-X⁶-X⁷-Arg-Lys-Gln-Met-Ala-Val-Lys-Lys-Tyr-Leu-X⁸-X⁹-X¹⁰-X¹¹-X¹²-X¹³-X¹⁴X¹⁵-X¹⁶-X¹⁷-X¹⁸-X¹⁹-X²⁰-X²¹-X²²; wherein X¹-X²² is any naturally occurring amino acid residue.

Preferred examples of suitable polypeptides of about 10 to 38 naturally occurring amino acid residues can include, without limitation, the following amino acid sequences: His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn (vasoactive intestinal peptide [VIP]); His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-Gly-Lys-Arg-Tyr-Lys-Gln-Arg-Val-Lys-Asn-Lys (pituitary adenylate cyclase-activating polypeptide [PACAP-38]); and His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu (PACAP-27).

Lung diseases or disorders that can be prevented and/or treated according to the method of the present invention can include, without limitation, COPD, preferably in conjunction with chronic bronchitis; chronic bronchitis not associated with COPD; lung disease not associated with pulmonary or arteriolar hypertension; or ARDS. Forms of COPD can include, without limitation, chronic bronchitis showing significant ventilation obstruction, pulmonary emphysema, or chronic cough, such as smoker's cough.

The pharmaceutical composition of the present invention can be administered daily, wherein such treatment results in an improvement of the FEV1 value by more than about 15% and an improvement of the paO2 value by more than about 35% after 3 months of daily treatment. The pharmaceutical composition can contain the effective polypeptide in a stabilized form, such as a pegylated form or in the form of a fusion protein, wherein the concentration of the effective polypeptide is between about 10 to 2000 μg/l, preferably between about 50 to 1500 μg/l, and most preferably between about 100 to 1000 μg/l. The pharmaceutical composition can be an aerosol, preferably based on an isotonic sodium chloride solution, which can be inhaled by a patient. The pharmaceutical composition thus can be used as a medicament or as a diagnostic means to evaluate pathological conditions in an individual.

In one embodiment of the present invention, a method is provided for improving or recovering the general state of health of an animal or human in need thereof which has been reduced by chronic bronchitis associated with a pathologically effective ventilation-perfusion mismatch (V/Q-mismatch) without significant obstructive ventilation disorder, by administering to the animal or human a pharmaceutically effective amount of a composition containing vasoactive intestinal peptide (VIP), pituitary adenylate cyclase-activating polypeptide (PACAP), or an analogous polypeptide having the same biological activity of VIP or PACAP.

It has been demonstrated that patients having a V/Q ratio in the pathological range of lower than about 0.8, preferably about 0.7; or higher than about 1.0, preferably about 1.1, after daily administration of the pharmaceutical composition of the present invention, will improve their V/Q ratio to between about 0.8 and 1.0, preferably between about 0.9 and 1.0.

As used herein, the term “animal” refers to a mammal, and preferably to a human.

As used herein, the phrase “same biological activity” is defined as the biological, physiological or therapeutic activity or functionality compared with the relevant properties of the peptides and polypeptides of the present invention, preferably VIP or PACAP.

As used herein, the term “derivative” is defined as a peptide compound which is derived more or less directly from the corresponding peptide, such as VIP or PACAP, and is altered by some additions, deletions, mutations or modifications without altering the biological properties of the parent peptide. Suitable VIP derivatives are, for example, disclosed in WO 8905857, WO 9106565, EP 0663406 and WO 9729126 (Fmoc-protected VIP), which are incorporated herein by reference. The term “derivative” also may include conjugates of peptides and polypeptides of the present invention which consist of the parent peptide or polypeptide coupled to lipophilic entities, such as liposomes, such as the VIP-liposome products which have improved properties with respect to bioavailability and proteolytic degradation disclosed in WO 9527496 or WO 9735561, which are incorporated herein by reference. Additionally, the term “derivative” may include fragments, and slightly modified truncated forms of fragments.

As used herein, the term “analogue” is defined as a compound which may have a different structure and composition compared to the polypeptides and peptides of the present invention, but without having altered biological properties, such as analogues of VIP. Preferably, VIP analogues of the present invention can be natural or synthetic peptides but also can be non-peptides. Examples of known VIP analogues are disclosed in EP 0325044 (cyclic peptides), EP 0225020 (linear peptides), EP 0536741 (cyclic VIP modifications), EP 0405242, EP 0184309 and EP 0613904, all of which are incorporated herein by reference. The term “analogue” also can include VIP homologues, which show great structural similarity to VIP. For example, one VIP homologue is PACAP and its truncated form PACAP-27. The term “analogue” also can include peptides and proteins and their homologues that can form amphipathic helices. Preferred VIP/PACAP homologues are peptides that comprise one or more consensus sequences. Examples are peptide histidine isoleucine (PHI), peptide histidine methionine (PHM), human growth hormone releasing factor (GRF), pituitary adenylate cyclase activating peptide (PACAP), secretin and glucagon.

As used herein, the phrase “stabilized form” is defined as a derivative or analogue wherein the parent peptide is altered in order to provide more stability and increased half-life in blood and serum. Such stabilized forms are preferred if the polypeptide is fragmented by enzyme activity. Possible stabilized forms are cyclic peptides or polypeptides like cyclic VIP or cyclic PACAP; fusion proteins, such as Fc-fusion proteins; or pegylated polypeptides, such as pegylated VIP or PACAP. Methods for manufacturing such polypeptides are well known in the art. Polypeptides and proteins may be protected against proteolysis by the attachment of chemical moieties, such as polyethylene glycol (PEG). Pegylation of polypeptides and proteins have been shown to protect against proteolysis (Sada et al., J. Fermentation Bioengineering, 71:137-139, 1991). Such chemical attachment can effectively block the proteolytic enzyme from physical contact with the protein backbone itself, and thus prevent degradation. In addition to protecting against proteolytic cleavage, chemical modification of biologically active proteins has been found to provide additional advantages under certain circumstances, such as increasing the stability and circulation time of the therapeutic protein and decreasing immunogenicity. (U.S. Pat. No. 4,179,337; Abuchowski et al., Enzymes as Drugs, J. S. Holcerberg and J. Roberts, eds. pp. 367-383, 1981; Francis, Focus on Growth Factors, 3: 4-10; EP 0 401 384). The addition of PEG increases the stability of the peptides and polypeptides of the present invention at physiological pH as compared to non-pegylated compounds. A pegylated polypeptide/protein also can be stabilized with regard to salts.

As used herein, the phrase “fusion protein” is defined as a compound, especially a stabilized form of a compound, consisting of a polypeptide of the present invention, preferably VIP or a VIP derivative or analogue, such as PACAP, which is fused to another peptide or protein. Such a fusion protein is preferably an immunglobulin (IgG) molecule, more preferably a fragment thereof, most preferably an Fc portion of an IgG molecule, preferably an IgG1. An Fc-VIP fusion protein is described in WO 200024278 (incorporated herein by reference) and shows an improved half-life in serum and blood. Other examples of fusion proteins are Fc-PACAP and FC-PACAP-27.

As used herein, the term “pharmaceutically acceptable carrier” is defined as an inert, non toxic solid or liquid filler, diluent or encapsulating material, which does not react adversely with the active compound or with the patient.

The pharmaceutical compositions of the present invention can contain one or more pharmaceutically acceptable carriers. Suitable carriers, such as liquid carriers, are well known in the art and can include, without limitation, sterile water, saline, aqueous dextrose, sugar solutions, ethanol, glycols and oils, such as petroleum, animal, vegetable, or synthetic oils. Exemplary oils can include, without limitation, peanut oil, soybean oil and mineral oil.

The formulations according to the present invention can be administered as unit doses containing conventional non-toxic pharmaceutically acceptable carriers, diluents, adjuvants and vehicles that are typical for parenteral administration.

The pharmaceutical compositions may be administered to the patient orally, in the form of tablets, pills, dragees, capsules, caplets, gels, syrups, slurries, suspensions and the like; parentally; or in form of aerosols for inhalation.

Tablets and capsules for oral administration can contain conventional excipients such as binding agents, fillers, diluents, tableting agents, lubricants, disintegrants, and wetting agents. The tablets can be coated according to methods well known in the art.

Parenteral administration can include, without limitation, subcutaneous, intravenous, intra-articular, intramuscular, intratracheal injection or infusion techniques. Parenteral compositions and combinations preferably are administered intravenously either in a bolus form or as a constant fusion according to known procedures.

Unit doses of the pharmaceutical composition administered according to the method of the present invention can contain daily required amounts of the compound according to the invention, or sub-multiples thereof to make up the desired dose. The optimum therapeutically acceptable dosage and dose rate for a given patient depends on a variety of factors, such as the activity of the specific compound administered, the age, body weight, general health, sex, diet, time and route of administration, rate of clearance, enzyme activity, and the object of the treatment, i.e., therapy or prophylaxis and the nature of the disease to be treated. Therefore, in compositions and combinations in a treated patient an effective therapeutic amount of the pharmaceutical composition of the present invention can be between about 5 ng to 28 μg/kg body weight, preferably between 15 ng to 25 μg/kg body weight, and most preferably between about 1 to 25 μg/kg body weight.

In another embodiment of the present invention, the pharmaceutical composition can be inhaled by the patient in the form of an aqueous solution that contains a water-soluble peptide having the biological and pharmacological activity of the above-described VIP, PACAP and related analogues, variants, derivatives, homologues and the like. The aqueous solution preferably is an isotonic saline solution which can contain additional drugs or other suitable ingredients. The aqueous solutions preferably contain the peptide compounds in a stabilized form, such as pegylated peptide compounds. The pharmaceutical composition of the present invention preferably is in the form of an aerosol for inhalation, especially when the patient is suffering from chronic bronchitis. Aerosols and techniques to make them are well known in the art. Administration by nasal spray techniques also are suitable.

The concentration of the particular peptide contained in the aqueous solutions can vary between about 10 to 2000 μg/L solution, preferably between about 150 to 1500 μg/L, and most preferably between about 100 to 1000 μg/L. If the particular peptide compound is in a stabilized form, such as pegylated VIP or pegylated PACAP, the concentration, as well as the overall dosage of the peptide compound, can be decreased. The peptides or polypeptides can be administered via inhalation about 3 to 4 times a day for about 3 to 20 minutes, preferably about 5 to 10 minutes, according to the severity of the disease and the potency of the compounds administered.

The present invention thus provides the new and unexpected finding that peptides or polypeptides that contain the highly conservative decapeptide sequence Arg-Lys-Gln-Met-Ala-Val-Lys-Lys-Tyr-Leu have high efficacy when administered to patients suffering from V/Q-mismatch lung disorders, preferably chronic bronchitis without obstruction of ventilation; COPD-related disorders that include chronic bronchitis; related lung diseases or disorders, such as unspecific chronic and/or irritating coughing; or symptoms which can be related to the above-described diseases or disorders and which preferably are not accompanied by lung hypertension, such as primary or secondary pulmonary hypertension (PPH, SPH). Surprisingly, it was found that compounds containing this decapeptide are highly active in patients suffering from the above-described diseases or disorders. Additionally, the peptides or polypeptides described herein are suitable for the prophylaxis and treatment of smoker's cough and similar symptoms.

It is believed that treating patients with the peptides and polypeptides of the present invention can provide great symptomatic relief as well as improve the general state of health of patients suffering from chronic V/Q-mismatch bronchitis and COPD-related chronic bronchitis and emphysema. For example, the forced expiratory volume (FEV) and the partial pressure of arterial oxygen (paO2) can be increased dramatically by about 10 to 50% within about two to five months in patients treated with VIP. In particular, the percentage increase of FEV1 varies between about 20 and 30% and the increase of paO2 varies between about 30 and 50% after approximately three months of treatment.

It is known that VIP is considered an effective treatment for asthma. The present invention provides, however, the new and unexpected finding that VIP and related compounds as defined herein have distinctly more efficacy in the treatment of COPD-related and V/Q-mismatch-related chronic bronchitis than in asthma. Interestingly, the peptides and polypeptides of the present invention do not act primarily like typical bronchodilatory drugs or anti-inflammatory drugs, such as corticosteroids, but have a different mode of action on pathologic bronchial tissue. Thus, VIP and related compounds not only are an alternative for generally known drugs used in this field, but also provide an improved pharmacological efficacy profile.

VIP is a 28 amino acid peptide hormone consisting of the following amino acid sequence: His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn. VIP and PACAP are peptides synthesized in various regions of the central nervous system as well as the peripheral nervous system, such as the hippocampus, cerebral cortex, pituitary gland and peripheral ganglia. In addition, VIP is secreted by immune cells and by some neoplastic cells (e.g. pancreatic cancer). VIP is thus widely distributed and mediates a variety of physiological responses including gastrointestinal secretion, relaxation of gastrointestinal vascular and respiratory smooth muscle, lipolysis in adipocytes, pituitary hormone secretion, and excitation and hyperthermia after injection into the central nervous system. Healthy individuals have relatively low concentrations of VIP in the circulation (<40 pg/ml serum).

Under physiologic conditions, VIP acts as a neuroendocrine mediator. Some recent findings suggest that VIP also regulates growth and proliferation of normal as well as malignant cells (Hultgard et al., Regul. Pept., 22, 267-274, 1988). Furthermore, VIP is a potent anti-inflammatory agent, as treatment with VIP has been shown to reduce significantly the incidence and severity of arthritis in an experimental model, completely abrogating joint swelling and destruction of cartilage and bone (Delgado et al., Nature Med., 7, 563-568, 2001). The biological effects of VIP are mediated via specific VIP receptors (VIP-R) located on the surface membranes of various cells (Ishihara, T. et al., Neuron, 8, 811-819, 1992). It has been suggested that VIP may exert stimulatory and trophic effects on neoplastic cells from neuroblastoma, breast, lung and colon cancer (Moody et al., Proc. Natl. Acad. Sci. USA, 90, 4345, 1993), by inducing its receptors via feedback mechanisms. It also has been shown that VIP produces dose-dependent stimulation of mitosis (Wollman et al., Brain Res., 624, 339, 1993). VIP and biologically functional analogues and derivatives thereof have been shown to have vascular smooth muscle relaxant activity (Maruno, K. et al., Am. J. Physiol. 268, L1047-L1051, 1995), hair growth activity, apoptosis activity, enhanced sustained bronchodilation activity without remarkable cardiovascular side effects, and are effective against disorders or diseases related to bronchial spasms including asthma, some cases of hypertension, impotence, ischemia, dry eye, and mental disorders, such as Alzheimer's disease (see e.g. WO 9106565, EP 0536741, U.S. Pat. No. 3,880,826, EP 0204447, EP 0405242, WO 9527496, EP 0463450, EP 0613904, EP 0663406, WO 9735561, EP 0620008). VIP also has been shown to decrease the resistance in the pulmonary vascular system (Hamasaki, Y. et al., J. Appl. Physiol., 54, 1607-1611, 1983; Iwabuchi, S., et al., Respiration, 64, 54-58, 1997; and Saga, T. et al., Trans. Assoc. Am. Physicians, 97, 304-310, 1984).

VIP receptors have been detected on airway epithelia of the trachea and the bronchioles. VIP receptors also are expressed in macrophages surrounding capillaries, in connective tissue of trachea and bronchi, in alveolar walls, and in the subintima of pulmonary veins and pulmonary arteries. Peptidergic nerve fibers are considered the source of VIP in the lungs (Dey, R. D. et al, Cell and Tissue Research, 220, 231-238, 1981; Said, S. I., Ann. N.Y. Acad. Sci. 629, 305-318, 1991). Other studies have shown a high rate of VIP-R expression in the lung, which is reflected in a high uptake of radiolabeled VIP in the lung of primary pulmonary hypertension (PPH) patients who are injected with 99mTc-VIP (Raderer, M., et al., Br. J. Cancer, 78, 1-5, 1998; Raderer, M., et al., J. Nucl. Med., 39, 1570-1575, 1998; Raderer, M., et al., J. Clin. Oncol., 18, 1331-1336, 2000; Virgolini, I. et al., J. Nucl. Med., 36, 1732-1739, 1995). Additionally, VIP and related compounds have been shown to be effective in the treatment of PPH, as well as secondary pulmonary hypertension (SPH) and arteriolar hypertension (PCT/EP01/13590).

PACAP is a 38 amino acid neuropeptide isolated from the ovine hypothalamus consisting of the following sequence: His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-Gly-Lys-Arg-Tyr-Lys-Gln-Arg-Val-Lys-Asn-Lys.

Two forms of the PACAP peptide have been identified: PACAP-38 and the C-terminally truncated PACAP-27. PACAP-27 shares 68 percent homology with VIP and has the following sequence: His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu.

PACAP is very potent in stimulating adenylate cyclase and thus increasing adenosine 3, 5-cyclic monophosphate (cAMP) in various cells. PACAP functions as a hypothalamic hormone, neurotransmitter, neuromodulator, vasodilator, and neurotrophic factor. PACAP has a major regulatory role in pituitary cells, apparently regulating gene expression of pituitary hormones and/or regulatory proteins that are responsible for controlling growth and differentiation of pituitary glandular cells. These regulatory effects appear to be exhibited directly and indirectly through a paracrine or autocrine action. PACAP also plays an important role in the endocrine system as a potent secretagogue for adrenaline from the adrenal medulla, as well as for stimulating the release of insulin. PACAP also displays a stage-specific expression in testicular germ cells during spermatogenesis, suggesting a regulatory role in the maturation of germ cells. In the ovary, PACAP is transiently expressed in the granulosa cells of the preovulatory follicles and appears to be involved in LH-induced cellular events, including prevention of follicular apoptosis. In the central nervous system, PACAP acts as a neurotransmitter and/or a neuromodulator. More importantly, PACAP is a neurotrophic factor that may play a significant role during the development of the brain. In the adult brain, PACAP appears to function as a neuroprotective factor that attenuates neuronal damage resulting from various insults. PACAP is widely distributed in the brain and peripheral organs, notably in the endocrine pancreas, gonads, and respiratory and urogenital tracts. Two types of PACAP binding sites have been characterized. Type I binding sites exhibit a high affinity for PACAP (and a much lower affinity for VIP), whereas type II binding sites have similar affinity for PACAP and VIP. Molecular cloning of PACAP receptors has shown the existence of three distinct receptor subtypes: a PACAP-specific PAC1 receptor, which is coupled to several transduction systems, and two PACAP/VIP-indifferent VPAC1 and VPAC2 receptors, which are primarily coupled to adenylyl cyclase. PAC1 receptors are particularly abundant in the brain and pituitary and adrenal glands, whereas VPAC receptors are expressed mainly in the lung, liver, and testes.

Compounds that contain the above-described highly conservative decapeptide sequence and have a total of 10 to 38, preferably 10 to 28 amino acid residues, have identical or very similar biological activity as VIP or PACAP, which also contain the highly conservative sequence. Furthermore, the peptides or polypeptides of the present invention preferably contain the additional sequence His-Ser-Asp and/or Phe-Thr-Asp, and most preferably contain the sequence His-Ser-Asp-X¹-X²-Phe-Thr-Asp-, which preferably is located at the N-terminal of the sequence, wherein X¹, X² may be any naturally occurring amino acid.

It is believed, without being bound by the theory, that VIP, PACAP and their truncated forms, for example PACAP-27, are highly active compounds for the prophylaxis and treatment of the above-described lung diseases or disorders due to their ability to inhibit and/or regulate cellular processes underlying these diseases in animals or humans.

In a further embodiment of the present invention, the pharmaceutical compounds can be administered to an animal or human in need thereof in combination with other pharmaceutically effective compounds, e.g., fast-acting beta2-agonists, such as albuterol; anticholinergic bronchodilators, such as ipratropium bromide; long-acting bronchodilators, inhaled or oral corticosteroids, antibiotics, or antiproliferative compounds, such as D-24851, Imatinib mesylate or guanylhydrazone CNI-1493.

It is believed that treatment with the pharmaceutical compositions of the present invention, alone or in combination with the above-described substances, will produce relatively few undesired side-effects in a subject in need of treatment.

The present invention is more particularly described in the following examples, which are intended to be illustrative only, because numerous modifications and variations therein will be apparent to those skilled in the art.

### EXAMPLE 1

Patient No. 1

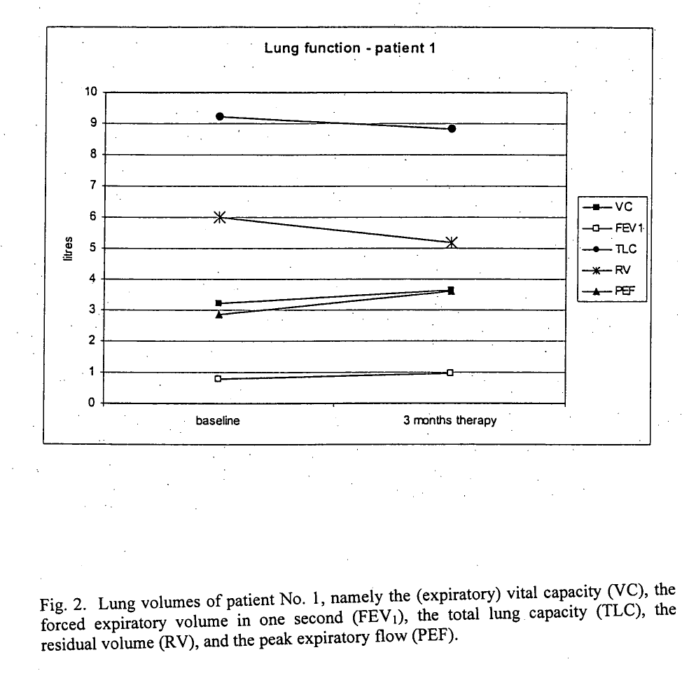

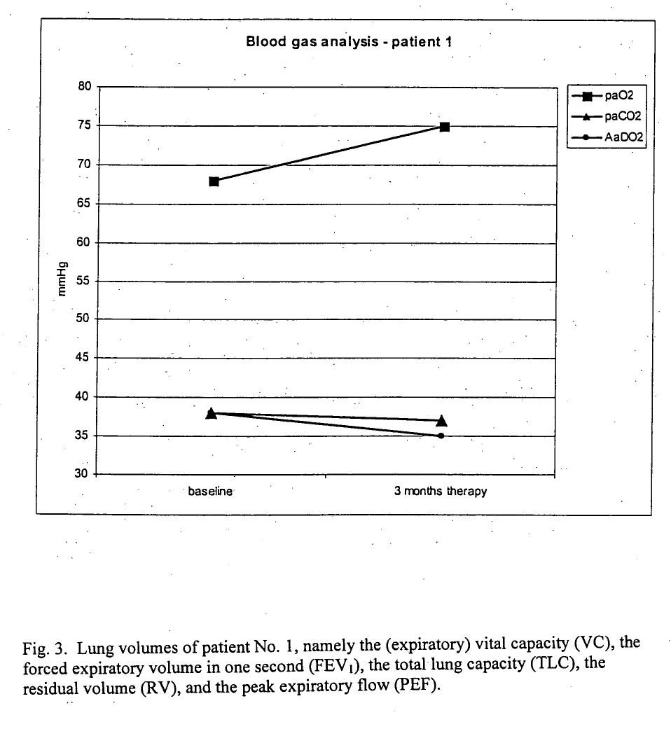

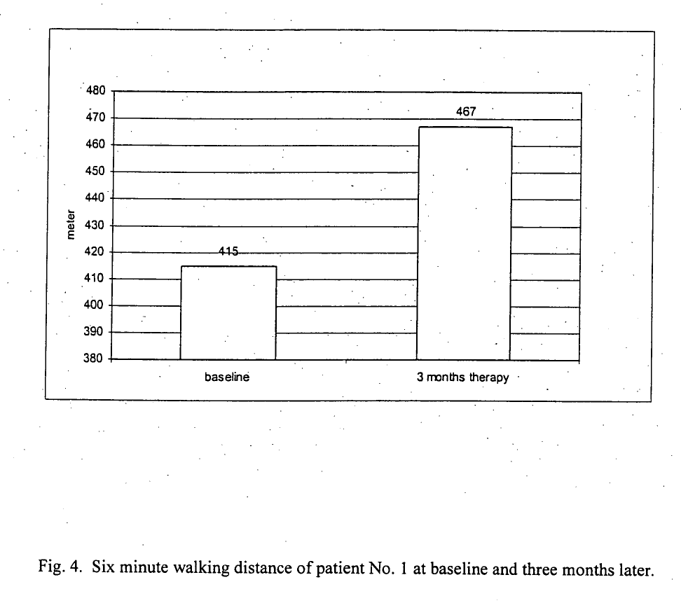

Patient No. 1 suffered from severe COPD with no sign of pulmonary hypertension. The patient inhaled VIP (200 μg in 3 ml NaCl 0.9%) for 15 minutes via the MicroDrop Master Jet (MPV, Truma, Germany) using a particle size of 3 μm to ensure alveolar deposition of the substance. Lung function parameters were measured at baseline (before inhalation of VIP) and after 3 months of therapy. FIG. 2 shows the lung volumes of patient No. 1, namely the (expiratory) vital capacity (VC), the forced expiratory volume in one second (FEV₁), the total lung capacity (TLC), the residual volume (RV), and the peak expiratory flow (PEF). FIG. 3 shows the blood gas analysis (paO2: partial arterial oxygen pressure; paCO2: partial arterial carbon dioxide pressure; and AaDO2: arterial-alveolar oxygen pressure difference) of patient No. 1 at baseline and three months later. FIG. 4 shows a six minute walking distance of patient No. 1 at baseline and three months later.

### EXAMPLE 2

Patient No. 2

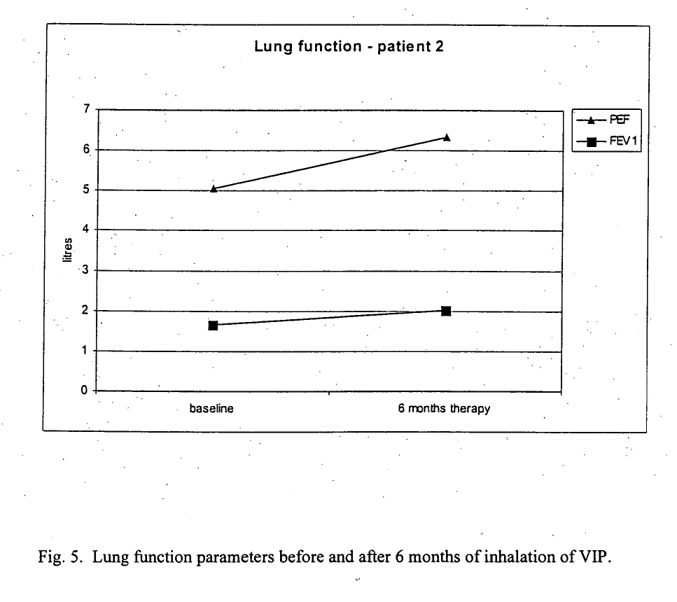

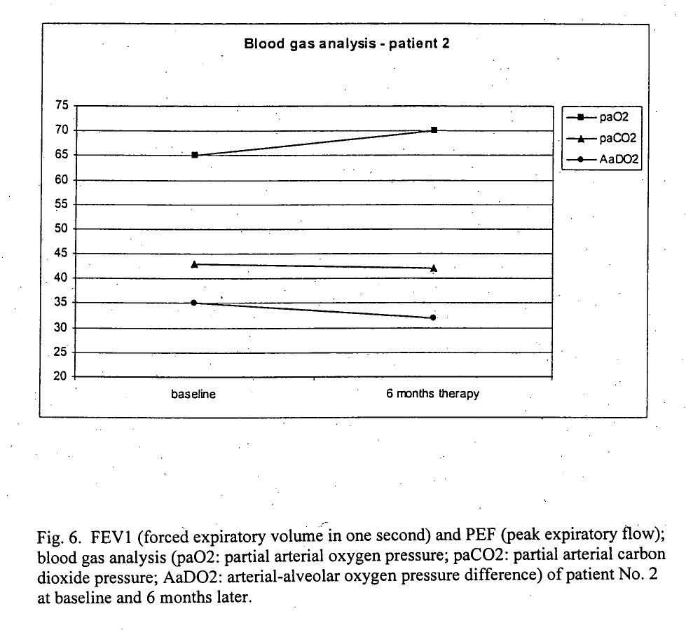

Patient No. 2 had severe COPD symptoms. The patient inhaled VIP (200 μg in 3 ml NaCl 0.9%) for 15 minutes via the MicroDrop Master Jet (MPV, Truma, Germany) using a particle size of 3 μm to provide alveolar deposition of the substance. Lung function parameters before and after 6 months of inhalation of VIP are given in FIG. 5. FEV1 (forced expiratory volume in one second) and PEF (peak expiratory flow); blood gas analysis (paO2: partial arterial oxygen pressure; paCO2: partial arterial carbon dioxide pressure; AaDO2: Arterial-alveolar oxygen pressure difference) of patient No. 2 at baseline and 6 months later are shown in FIG. 6.

### EXAMPLE 3

Patient No. 3

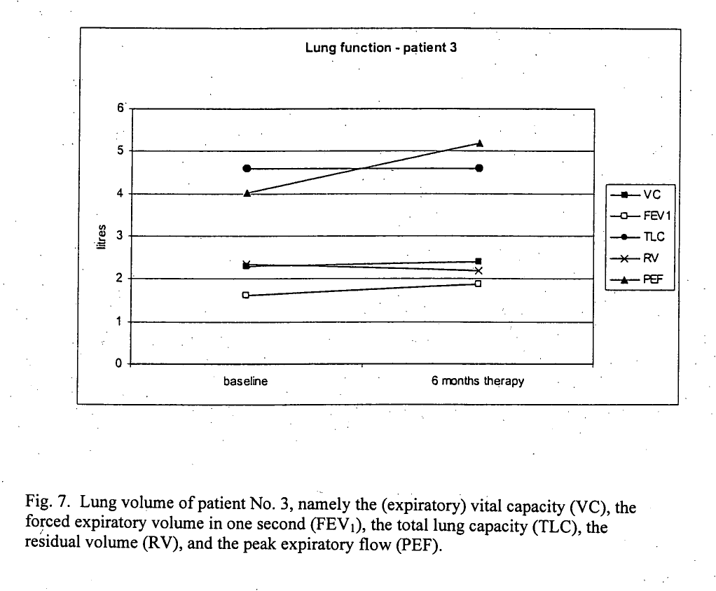

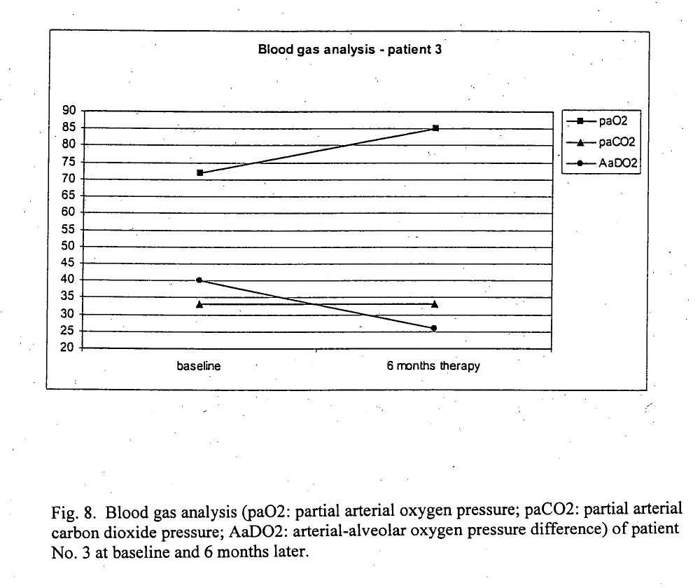

Patient No. 3 also suffered from severe COPD with no sign of pulmonary hypertension. The patient inhaled VIP (200 μg in 3 ml NaCl 0.9%) for 15 minutes via the MicroDrop Master Jet (MPV, Truma, Germany) using a particle size of 3 μm to ensure alveolar deposition of the substance. FIG. 7 shows the lung volume of patient No. 3, namely the (expiratory) vital capacity (VC), the forced expiratory volume in one second (FEV₁), the total lung capacity (TLC), the residual volume (RV), and the peak expiratory flow (PEF). Lung function parameters were measured at baseline (before inhalation of VIP) and after 6 months of therapy. FIG. 8 shows the blood gas analysis (paO2: partial arterial oxygen pressure; paCO2: partial arterial carbon dioxide pressure; AaDO2: arterial-alveolar oxygen pressure difference) of patient No. 3 at baseline and 6 months later.

### EXAMPLE 4

Patient No. 4

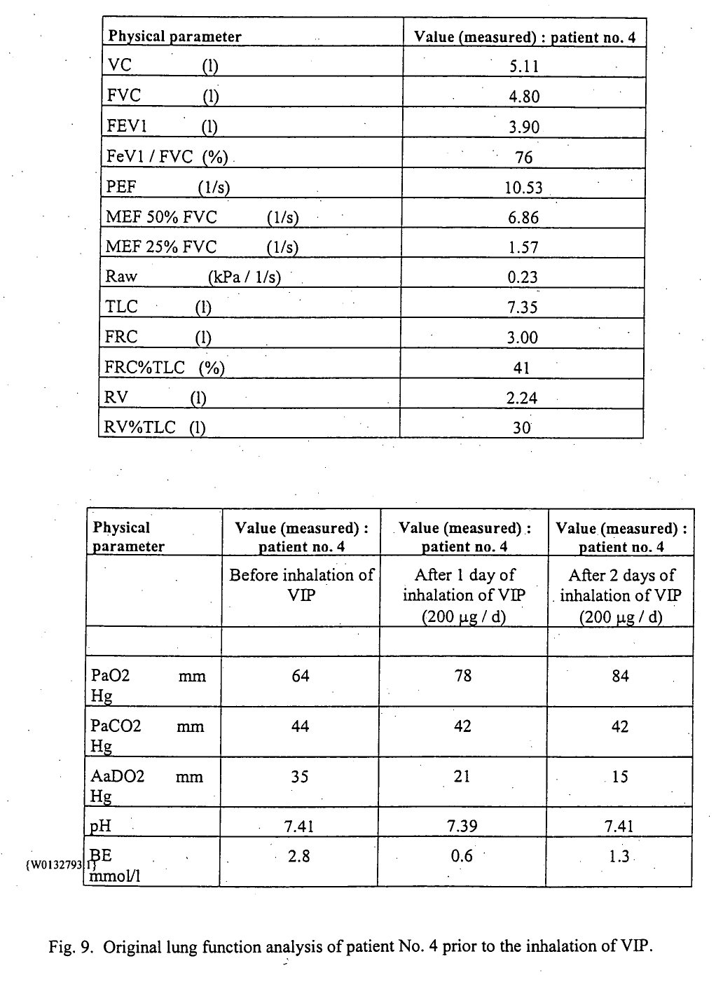

Patient No. 4 suffered from an acute worsening of long-term bronchitis, but demonstrated no bronchial obstruction (FEV1 before inhalation of VIP: 84%). FIG. 9 shows the original lung function analysis of patient No. 4 prior to the inhalation of VIP. The V/Q-mismatch due to peripheral lung inflammation caused a severe decrease of paO2 that was significantly ameliorated by VIP after 1 and 2 days of inhalation, respectively, after which lung function analysis demonstrated completely normal pulmonary gas exchange, thus demonstrating that the V/Q-mismatch had been totally removed.

All of the above examples demonstrate that the peptides and polypeptides of the present invention have beneficial effects in the treatment preferably of chronic bronchitis without obstructive ventilation pattern but with a V/Q-mismatch, and COPD. These data show a dramatic improvement in diseases that heretofore have been insufficiently treated. Indeed, all of the peptides and polypeptides containing the highly conservative above-described decapeptide sequence were very efficacious.

While the invention has been particularly shown and described with reference to preferred embodiments thereof, it will be understood by those skilled in the art that various alterations in form and detail may be made therein without departing from the spirit and scope of the invention.

1. A method for preventing and/or treating a lung disease or disorder that is associated with a pathologically effective ventilation-perfusion (V/Q) mismatch in an animal or human in need thereof, comprising administering to the animal or human in unit dosage form a therapeutically effective amount of a pharmaceutical composition comprised of a carrier and a polypeptide of 10 to 38 naturally occurring amino acid residues that contain the sequence Arg-Lys-Gln-Met-Ala-Val-Lys-Lys-Tyr-Leu.

2. The method of claim 1, wherein the V/Q ratio of the animal or human before starting treatment with said pharmaceutical composition is less than 0.8 or greater than 1.0.

3. The method of claim 1, wherein the polypeptide consists of 18 to 38 naturally occurring amino acid residues and has an N-terminal starting sequence of His-Ser-Asp-X¹-X²-Phe-Thr-Asp-, wherein X¹ and X² may be any naturally occurring amino acid residue.

4. The method of claim 1, wherein the polypeptide is selected from the group consisting of Arg-Lys-Gln-Met-Ala-Val-Lys-Lys-Tyr-Leu; Phe-Thr-Asp-X¹-X²-X³-X⁴-X⁵-Arg-Lys-Gln-Met-Ala-Val-Lys-Lys-Tyr-Leu-Asn-Ser-Ile-Leu-Asn; Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn; Phe-Thr-Asp-Ser-Tyr-Ser-Arg-Tyr-Arg-Lys-Gln-Met-Ala-Val-Lys-Lys-Tyr-Leu; His-Ser-Asp-X¹-X²-Phe-Thr-Asp-X³-X⁴-X⁵-X⁶-X⁷-Arg-Lys-Gln-Met-Ala-Val-Lys-Lys-Tyr-Leu; His-Ser-Asp-Ala-Val-Phe-Thr-Asp-Asn-Tyr-Thr-Arg-Leu-Arg-Lys-Gln-Met-Ala-Val-Lys-Lys-Tyr-Leu; His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu; His-Ser-Asp-X¹-X²-Phe-Thr-Asp-X³-X⁴-X⁵-X⁶-X⁷-Arg-Lys-Gln-Met-Ala-Val-Lys-Lys-Tyr-Leu-X⁸-X⁹-X¹⁰-X¹¹ (-X¹²); His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn (VIP); His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu (PACAP-27); His-Ser-Asp-X¹-X²-Phe-Thr-Asp-X³-X⁴-X⁵-X⁶-X⁷-Arg-Lys-Gln-Met-Ala-Val-Lys-Lys-Tyr-Leu-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²; and His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-Gly-Lys-Arg-Tyr-Lys-Gln-Arg-Val-Lys-Asn-Lys (PACAP-38), wherein X¹-X²² is any naturally occurring amino acid residue.

5. The method of claim 1, wherein the polypeptide is vasoactive intestinal peptide (VIP), having the sequence: His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn; or pituitary adenylate cyclase-activating polypeptide (PACAP), said PACAP having the following two sequences: His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-Gly-Lys-Arg-Tyr-Lys-Gln-Arg-Val-Lys-Asn-Lys (PACAP-38); and His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu (PACAP-27), or an analogous polypeptide of VIP or PACAP, such as a derivative, variant, fragment or homologue, that has the same biological activity of VIP or PACAP.

6. The method of claim 5, wherein the polypeptide is a homologue of VIP or PACAP, said homologue comprising one or more consensus sequences of VIP or PACAP.

7. The method of claim 6, wherein the homologue is selected from the group consisting of peptide histidine isoleucine (PHI), peptide histidine methionine (PHM), human growth hormone releasing factor (GRF), pituitary adenylate cyclase activating peptide (PACAP), secretin and glucagon.

8. The method of claim 1, wherein the lung disease or disorder is selected from the group consisting of COPD; COPD in conjunction with chronic bronchitis; chronic bronchitis not associated with COPD; lung disease not associated with pulmonary or arteriolar hypertension; and ARDS.

9. The method of claim 8, wherein the COPD is selected from the group consisting of chronic bronchitis showing significant ventilation obstruction, pulmonary emphysema and chronic cough, such as smoker's cough.

10. The method of claim 1, wherein the lung disease or disorder is chronic bronchitis that is not associated with any significant obstructive ventilation disorder.

11. The method of claim 1, wherein the pharmaceutical composition is administered daily, said daily administration improving FEV1 values by more than about 15% and paO₂ values by more than about 35% after about three months of treatment.

12. The method of claim 1, wherein said pharmaceutical composition contains said polypeptide in a stabilized form.

13. The method of claim 12, wherein the stabilized form of the polypeptide includes cyclic polypeptides, fusion proteins, such as Fc-fusion proteins, or pegylated polypeptides.

14. The method of claim 1, wherein the carrier is inert and non-toxic and selected from the group consisting of solid fillers, liquid fillers, diluents and encapsulating materials.

15. The method of claim 14, wherein the liquid carrier is selected from the group consisting of sterile water, saline, aqueous dextrose, sugar solutions, ethanol, glycols and oils, such as petroleum, animal, vegetable or synthetic oils.

16. The method of claim 1, wherein the route of administration of the pharmaceutical composition is oral, parenteral or nasal.

17. The method of claim 16, wherein the form of the oral administration is selected from the group consisting of tablets, pills, dragees, capsules, caplets, gels syrups, slurries and suspensions.

18. The method of claim 17, wherein the tablets and capsules for oral administration can contain conventional excipients selected from the group consisting of binding agents, fillers, diluents, tableting agent, lubricants, disintegrants and wetting agents.

19. The method of claim 16, wherein the parenteral administration is selected from the group consisting of subcutaneous, intravenous, intra-articular, intramuscular, intratracheal and infusion.

20. The method of claim 16, wherein the pharmaceutical composition is administered nasally.

21. The method of claim 20, wherein the nasal administration is in the form of an aerosol.

22. The method of claim 14, wherein the aerosol is an isotonic sodium chloride aqueous solution containing said polypeptide in a pegylated form.

23. The method of claim 22, wherein the pharmaceutical composition is administered nasally 3 to 4 times a day, each administration lasting for about 3 to 20 minutes.

24. The method of claim 22, wherein the pharmaceutical composition is administered nasally 3 to 4 times a day, each administration lasting for about 5 to 10 minutes.

25. The method of claim 22, wherein the concentration of said polypeptide in the aerosol is between about 10 to 2000 μg/L.

26. The method of claim 22, wherein the concentration of said polypeptide in the aerosol is between about 50 to 1500 μg/L.

27. The method of claim 22, wherein the concentration of said polypeptide in the aerosol is between about 100 to 1000 μg/L.

28. The method of claim 1, wherein the therapeutically effective dose is between about 5 ng to 28 μg/kg body weight.

29. The method of claim 1, wherein the therapeutically effective dose is between about 15 ng to 25 μg/kg body weight.

30. The method of claim 1, wherein the therapeutically effective dose is between about 1 to 25 μg/kg body weight.

31. A method for improving or recovering the general state of health in an animal or human which has been reduced by chronic bronchitis associated with a pathologically effective ventilation-perfusion mismatch (V/Q-mismatch) but without significant obstructive ventilation disorder, comprising administering to the animal or human in unit dosage form a therapeutically effective amount of a pharmaceutical composition containing a carrier and VIP, PACAP, or an analogous polypeptide having the same biological activity.

32. The method of claim 31, wherein the pharmaceutical composition is an aerosol comprising said polypeptide in a concentration range of between about 100 to 1000 μg/L.

33. A method for reducing or eliminating V/Q-mismatch that is not associated with COPD in the lung of a diseased animal or human, comprising administering to the animal or human in unit dosage form a therapeutically effective amount of a pharmaceutical composition containing a carrier and VIP, PACAP, or an analogous polypeptide having the same biological activity.

34. A method for preventing and/or treating a lung disease or disorder that is associated with a pathologically effective V/Q-mismatch in an animal or human in need thereof, comprising administering to the animal or human in unit dosage form a therapeutically effective amount of a pharmaceutical composition in combination with other pharmaceutically effective compounds, said pharmaceutical composition containing a carrier and a polypeptide of 10 to 38 naturally occurring amino acid residues that contain the sequence Arg-Lys-Gln-Met-Ala-Val-Lys-Lys-Tyr-Leu in combination with other pharmaceutically effective compounds.

35. The method of claim 34, wherein the other pharmaceutically effective compounds are selected from the group consisting of fast-acting beta2-agonists, such as albuterol; anticholinergic bronchodilators, such as ipratropium bromide; long-acting bronchodilators; inhaled or oral corticosteroids; antibiotics; and antiproliferative compounds, such as D-24851, Imatinib mesylate or guanylhydrazone CNI-1493.


SEQUENCE LISTING (14 sequences)

SEQ ID NO: 1 - protein, 28 aa, Homo sapiens
  misc_feature (1)..(28): vasoactive intestinal peptide (VIP
HSDAVFTDNYTRLRKQMAVKKYLNSILN

SEQ ID NO: 2 - protein, 38 aa, Homo sapiens
  pituitary adenylate cyclase-activating polypeptide PACAP-38 (1)..(38)
HSDGIFTDSYSRYRKQMAVKKYLAAVLGKRYKQRVKNK

SEQ ID NO: 3 - protein, 27 aa, Homo sapiens
  MISC_FEATURE (1)..(27): truncated PACAP
HSDGIFTDSYSRYRKQMAVKKYLAAVL

SEQ ID NO: 4 - protein, 8 aa, Artificial
  derived from VIP / PACAP
HSDXXFTD

SEQ ID NO: 5 - protein, 10 aa, Artificial
  derived from VIP / PACAP
RKQMAVKKYL

SEQ ID NO: 6 - protein, 10 aa, Artificial
  derived from VIP / PACAP
RKQMAVKKYL

SEQ ID NO: 7 - protein, 23 aa, Artificial
  derived from VIP / PACAP
FTDXXXXXRKQMAVKKYLNSILN

SEQ ID NO: 8 - protein, 23 aa, Artificial
  derived from VIP / PACAP
FTDNYTRLRKQMAVKKYLNSILN

SEQ ID NO: 9 - protein, 18 aa, Artificial
  derived from VIP / PACAP
FTDSYSRYRKQMAVKKYL

SEQ ID NO: 10 - protein, 23 aa, Artificial
  derived from VIP / PACAP
HSDXXFTDXXXXXRKQMAVKKYL

SEQ ID NO: 11 - protein, 23 aa, Artificial
  derived from VIP / PACAP
HSDAVFTDNYTRLRKQMAVKKYL

SEQ ID NO: 12 - protein, 23 aa, Artificial
  derived from VIP / PACAP
HSDGIFTDSYSRYRKQMAVKKYL

SEQ ID NO: 13 - protein, 28 aa, Artificial
  derived from VIP / PACAP
HSDXXFTDXXXXXRKQMAVKKYLXXXXX

SEQ ID NO: 14 - protein, 38 aa, Artificial
  derived from VIP/PACAP
HSDXXFTDXXXXXRKQMAVKKYLXXXXXXXXXXXXXXX